(19) United States
(12) Patent Application Publication
(10) Pub. No.: US 2026/0278760 A1
(43) Pub. Date: Sep. 17, 2026

(54) Methods, systems, and computer-readable media for assessing meat quality

(71) Applicant: MEQ PROBE PTY LTD, Victoria (AU)

(72) Inventors: Jordan YEOMANS, Victoria (AU); Mia ATCHESON, Victoria (AU); Remo CARBONE, Victoria (AU); Lawrence AU, Victoria (AU)

(21) Appl. No.: 19/469,900

(22) PCT Filed: Mar. 28, 2024
(86) PCT No.: PCT/AU2024/050295

§ 371 Date: Sep. 26, 2025

(30) Foreign Application Priority Data
Mar. 28, 2023 (AU) 2023900869

(51) Int. Cl. G06T 7/00; G06T 7/11

(52) U.S. Cl. CPC G06T 7/0002; G06T 7/11; G06T 2207/10016; G06T 2207/30128; G06T 2207/30168

(57) ABSTRACT

Described embodiments relate to methods of determining quality assessment measures of carcases. The method comprises determining video data comprising a sequence of frames, wherein at least some of the frames depict a part of a carcase to be quality assessed. For a first frame of the sequence of frames, the method comprises a) determining a frame suitability score for assessing a first trait; b) responsive to the frame suitability score being greater than a threshold frame suitability score for the first trait, determining the frame as a suitable frame for assessing the first trait; c) determining, by a segmentation model, a region of interest in the frame for assessing the first trait; d) determining, by a first trait prediction model, a prediction score for the first trait based on the determined region of interest; and e) determining an assessment confidence rating for the first trait. The method further comprises responsive to determining that the assessment confidence rating for the first trait has not exceeded the assessment confidence threshold for the first trait, performing steps a) to e) for a subsequent frame in the sequence; and responsive to determining that the assessment confidence rating for the first trait has exceeded the assessment confidence threshold for the first trait, determining a quality assessment measure of the first trait based on the prediction score for the first trait of the determined suitable frames.

### TECHNICAL FIELD

Described embodiments generally relate to methods, systems, and computer-readable media for assessing meat quality of animal carcases, such as cold carcases.

### BACKGROUND

In the field of assessment of meat quality, carcases are typically assessed by inspection to determine the quality of the meat they contain.

Existing techniques used to assess meat quality tend to involve capturing an image of an assessment region of the carcase using a specific technical imaging device made for assessing meat quality.

It is desired to address or ameliorate one or more shortcomings or disadvantages associated with such prior art, or to at least provide a useful alternative hereto.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### SUMMARY

Some embodiments relate to a method comprising: determining video data comprising a sequence of frames, wherein at least some of the frames depict a part of a carcase to be quality assessed; for a first frame of the sequence of frames: a) determining a frame suitability score for assessing a first trait; b) responsive to the frame suitability score being greater than a threshold frame suitability score for the first trait, determining the frame as a suitable frame for assessing the first trait; c) determining, by a segmentation model, a region of interest in the frame for assessing the first trait; d) determining, by a first trait prediction model, a prediction score for the first trait based on the determined region of interest; and e) determining an assessment confidence rating for the first trait; responsive to determining that the assessment confidence rating for the first trait has not exceeded the assessment confidence threshold for the first trait, performing steps a) to e) for a subsequent frame in the sequence; and responsive to determining that the assessment confidence rating for the first trait has exceeded the assessment confidence threshold for the first trait, determining a quality assessment measure of the first trait based on the prediction score for the first trait of the determined suitable frames.

The method may further comprise determining the assessment confidence rating for the first trait may comprise: determining one or more of: (i) a number of determined suitable frames for the first trait; and (ii) a function of the frame suitability scores for the first trait of the determined suitable frames.

The method may further comprise determining the assessment confidence rating for the first trait may comprise: determining a function of the prediction scores for the first trait of the determined suitable frames. Wherein determining a function of the prediction scores for the first trait of the determined suitable frames may comprise determining an arithmetic mean taken over the interval: [Bₘᵢₙ, Bₘₐₓ], where Bₘᵢₙ is defined by the function:

$${B}_{min} = {P}_{S} - ( {tol}_{IQR} \times IQR )$$

and where Bₘₐₓ is defined by the function:

$${B}_{max} = {P}_{A} + ( {tol}_{IQR} \times IQR )$$

where P_{A} is a prediction value of a first percentile of prediction scores of the first trait; where P_{B} is a prediction value of a second percentile of prediction scores of the first trait; where tol_{IQR} is a tolerance parameter; and where IQR is an inter quartile range defined by IQR=P_{A}−P_{B}.

The method may further comprise excluding the lowest p_{drop} percentage of prediction scores for one or more of: (i) a measure of lack of glare; (ii) a measure of sharpness; and (iii) a measure of segmentation size; where p_{drop} is a configurable parameter. The method may further comprise: determining a frame number threshold for the sequence of frames; responsive to determining the number of frames exceeds the frame number threshold, discarding the frame suitability score, prediction score, and confidence rating for the earliest determined frame. The first percentile may be a smaller percentile than the second percentile.

The method may further comprise: responsive to determining the frame as a suitable frame for assessing the first trait, performing steps c) and d); and responsive to determining the frame as not being a suitable frame for assessing the first trait, omitting steps c) and d).

Determining video data may comprise receiving a video stream.

The frame suitability score may be based on one or more of: (i) a measure of lack of glare, (ii) a measure of sharpness, (iii) a measure of segmentation size; (iv) a measure of segmentation margin of the region of interest; (v) a measure of segmentation roundness of the region of interest.

Determining the frame suitability score may comprise determining the frame suitability score according following equation:

$$\begin{matrix}{S}_{t} = {\sum }_{r} & max ( {v}_{r} {a}_{r}^{t} + {b}_{{r}^{′}}^{t}\end{matrix} , 0 ) ,$$

where Sₜ is the suitability score; vᵣ is a value of rating-type r for the first frame;

$${a}_{r}^{t}$$

is a scaling coefficient of the rating type r for trait t; and

$${b}_{r}^{t}$$

is an offset coefficient of rating r for trait t.

The method may further comprise: outputting the quality assessment measure of the first trait to a user interface.

The first trait may comprise any one of: marbling; marbling fineness; ribeye area; rib fat proportion; intramuscular fat; fat colour; and meat colour.

The method may further comprise: for the first frame of the sequence of frames: f) determining a frame suitability score for assessing a second trait, wherein the second trait is different from the first trait; g) responsive to the frame suitability score being greater a threshold frame suitability score for the second trait, determining the frame as a suitable frame for assessing the second trait; h) determining, by a second segmentation model, area region of interest in the frame for assessing a second trait; i) determining, by a second trait prediction model, a prediction score for the second trait based on the determined region of interest; and j) determining an assessment confidence rating for the second trait; responsive to determining that the assessment confidence rating for the second trait has not exceeded the assessment confidence threshold for the second trait, performing steps a) to e) for a subsequent frame in the sequence; and responsive to determining that the assessment confidence rating for the second trait has exceeded the assessment confidence threshold for the second trait, determining a quality assessment measure of the second trait based on the prediction scores for the second trait of the determined suitable frames. The second trait may be different from the first trait and comprises any one of: marbling; marbling fineness, ribeye area; rib fat thickness; intramuscular fat; fat colour, and meat colour.

The frame suitability score may be based on one or more frame suitability ratings, each frame suitability rating being indicative of the suitability of a frame with respect to a specific measure, the method further comprising, for each of the one or more frame suitability ratings: comparing the frame suitability rating with a respective measure specific rating threshold; and responsive to determining that the frame suitability rating does not meet the threshold, determining the frame as unsuitable and excluding the frame from further processing.

In some embodiments, the step of determining, by a segmentation model, a region of interest in the frame for assessing the first trait, includes: receiving a point cloud and isolating the area of the point cloud corresponding to the region of interest; determining a plane, fitted to points of the point cloud; projecting the points of the point cloud onto the plane to generate a plurality of projected planar points; rasterizing the projected planar points to a high-resolution image where each pixel cell of the high-resolution image corresponds to a region of the plane of a real world area; converting the high-resolution image to a binary image; applying a morphological operation to fill in holes in the binary image; identifying the contour of the region of interest in the binary image; and responsive to only a single contour being identified, converting the area inside the contour to a real-world area estimate of the region of interest.

Some embodiments relate to a meat assessment system, comprising: at least one processor; memory accessible to the at least one processor and comprising computer executable instructions, which when executed by the at least one processor, causes the system to perform the described method.

The system may further comprise: a user interface configured to display the determined quality assessment measure for the first trait. The system may further comprise a video capture device to capture video data, controlled by the at least one processor.

Some embodiments relate to a non-transitory machine-readable medium storing instructions which, when executed by one or more processors, cause an electronic apparatus to perform the described method.

### BRIEF DESCRIPTION OF DRAWINGS

FIG. 1 is an example schematic of an animal product undergoing meat quality assessment, according to some embodiments;

FIG. 2 is a block diagram of an analysis device for performing meat quality assessment, according to some embodiments;

FIG. 3 is a flowchart of a method of assessing meat quality, according to some embodiments; and

FIGS. 4A and 4b are example screenshots of displays of a user interface of the analysis device of FIG. 2, according to some embodiments.

### DETAILED DESCRIPTION

Described embodiments generally relate to methods, systems, and computer-readable media for assessing meat quality of animal carcases, such as cold carcases.

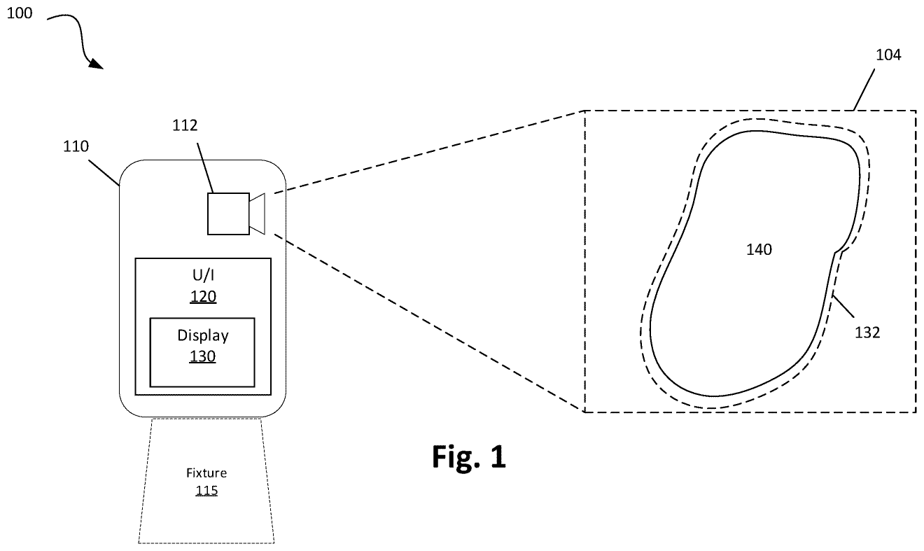

In meat processing environments, it is often beneficial to quickly and reliably grade meat carcases for further processing. FIG. 1 is an example schematic of a meat product 140 undergoing meat quality assessment using an analysis device 110, according to some embodiments. The analysis device 110 is configured to assess the meat product 140 to determine a quality assessment of one or more traits or characteristics of the meat product 140. For example, the traits may include one or more of marbling, marbling fineness, ribeye area, rib fat thickness, intramuscular fat proportion, meat colour, fat colour, and eye muscle area (EMA). Eye muscle area may be determined as a measure of the rib-eye muscle in units squared, for example, cm². Rib fat thickness may refer to the absolute-real world rib fat thickness of the meat product 140. In some embodiments, rib fat thickness may include a proportional value comparative to another aspect of the geometry of a cut surface of the meat product 140. In some embodiments, marbling fineness may include a measure of how small and/or evenly distributed the fat particles are within the EMA. Intramuscular fat proportion may also be referred to as intramuscular fat or IMF.

Generally, in the described embodiments, a user using the analysis device 110 captures video data of the meat product 140 using the analysis device 110. For example, the analysis device 110 may comprise a video capture component 112. The video data comprises a sequence of frames 104, each frame depicting a view or image of the meat product 140. The analysis conducted by the analysis device 110 may be conducted simultaneously, or in near real time, as the capture of the video data.

The analysis device 110 is configured to analyse frames of the video data to determine quality assessment measure(s) for the trait(s).

The quality assessment measure(s) for the trait(s) may be determined using machine learning (ML) model(s), such as neural networks. The model(s) determine prediction score(s) for respective trait(s) for each frame of a plurality of captured frames. A frame suitability score may be determined for each frame to determine whether the frame is suitable for use in assessing the meat quality. In some embodiments, determination of the suitability of a frame, and determination of prediction score(s) for respective trait(s) for the frame are performed in parallel, or substantially simultaneously.

The analysis device 110 may determine an assessment confidence rating to determine whether a quality assessment measure should be determined based on the information acquired. For example, the assessment confidence rating may depend on whether a sufficient number of frames have been acquired (for example, at least ten), and/or whether a sufficient number of suitable frames have been acquired, and/or whether a function of the prediction scores for the suitable frames meet a threshold, for example, are sufficiently high or sufficiently low.

Once the analysis device 110 determines quality assessment measure(s) for the trait(s), the analysis device 110 provides or outputs the determined quality assessment measure(s) to a user interface 120 of the analysis device 110. For example, the analysis device 110 may present the determined quality assessment measure(s) on a display screen 130 of the user interface 120, thereby allowing a user to see the determined quality measure of the meat product 140 with respect to each trait. The analysis device 110 may store the determined quality assessments measure(s) in memory 210. The analysis device 110 may send determined quality assessment measures(s) to an external device(s) via communications interface 226.

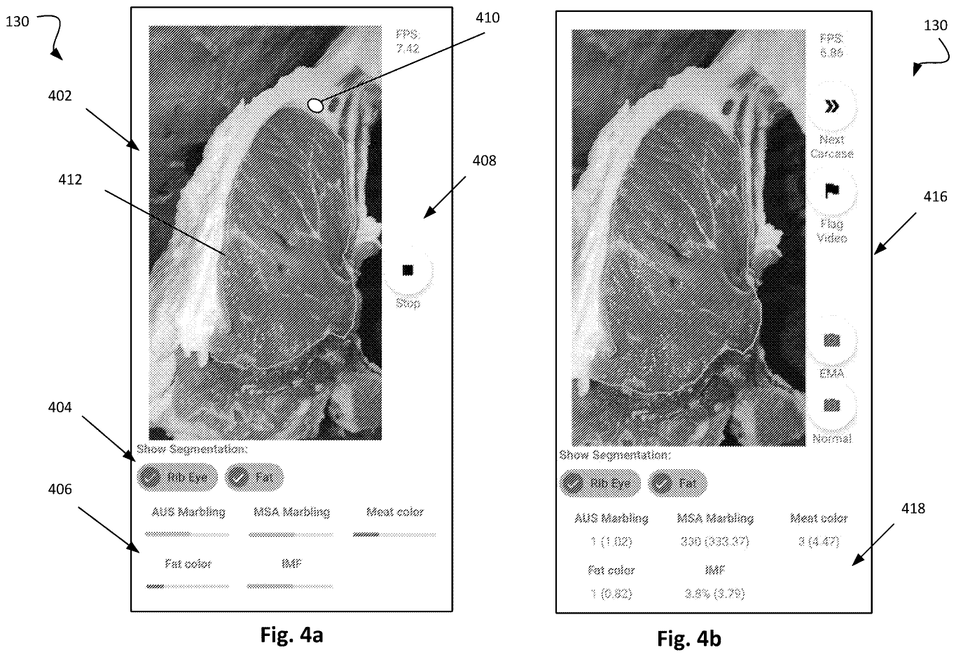

Examples of screenshots of a display screen 130 of the user interface 120 of the analysis device 110 are shown in FIGS. 4A to 4b, as discussed in more detail below.

Performing the meat quality assessment of the meat product 140 using video may allow for a high degree of accuracy and/or efficiency. As the assessment method is performed on a series of frames 104, data may be acquired relatively quickly, and/or the determination of the desired traits may have improved accuracy as it is determined from an array or plurality of frames 104. This may be preferable to using single or individual images, for example, as single images may be limited by factors such as environmental lighting, lack of sharpness, glare, and/or other factors which may impact the ability to make an accurate meat quality assessment from the single image. Furthermore, to capture a high quality single image, it is often necessary to pause the operation of a meat processing line, which can contribute to costly delays in terms of refrigeration time and/or processing output. In contrast, performing an assessment on more than one frame of a set of images or video may provide the advantage of allowing operators to assess meat quality in a variety of lighting and/or imaging conditions without significantly impacting the accuracy and/or efficiency of the assessment.

The described method may provide for an efficient, and/or reliable technique for assessing meat quality. The ability to assess a video stream, which comprises a sequence of images or frames 104, means that a relatively large amount of data may be captured in a short space of time for analysis. The determination of frame suitability on an individual or “frame by frame” basis means that operators may not require special training to operate a camera, for example, to accurately place a camera for image capture, as may be the case where assessment is performed on single images. Accordingly, the benefits of using trained machine learning model(s) of the meat quality assessment module 214 mean that operators may be able to capture video of the general area and rely on the functionality of the meat quality assessment module 214 to assess which parts of the video are not suitable for analysis. This approach may have significant benefit in an abattoir, for example, where the environmental conditions such as ambient lighting cannot be controlled, and in some instances the carcases may be moving as they are graded. Furthermore, the described systems and methods may be of particular benefit to further processing plants, which can benefit from the reliable and/or efficient determination of meat quality in the products they receive for further processing. The described systems and methods may be of particular benefit to retailers, to identify meat quality at an individual cut level. Furthermore, the described systems and methods may be of benefit in consumer applications to allow for easy-to-use grading of purchased meat products, evaluation of value of the meat product, and/or to aid in assessing optimal cooking times and/or procedures.

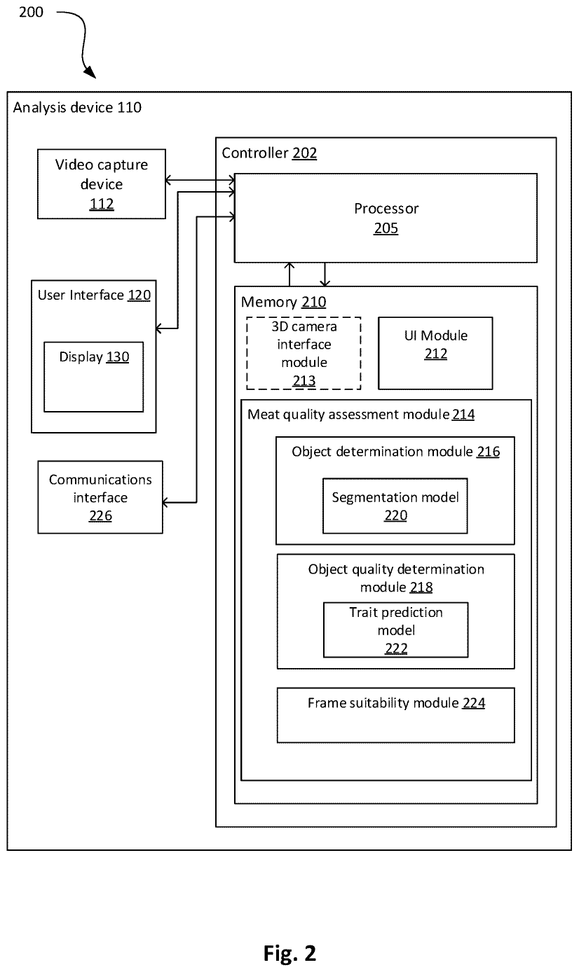

FIG. 2 depicts the analysis device 110, according to some embodiments. As illustrated, the analysis device 110 may comprise a video capture device 112 for capturing video data of a meat product to be assessed. The analysis device 110 may comprise a user interface 120 for outputting, to a user for example, determined quality assessment measure(s) for the trait(s) of the meat product. The analysis device 110 may be affixed to a support 115.

In some embodiments, the analysis device 110 may be a smartphone, such as a Samsung Galaxy™ model smartphone. In such embodiments, the video capture device 112 may comprise a digital camera, in-built within the smartphone. In other embodiments, the video capture device 112 may be a separate unit installed remotely from the controller 202. In such embodiments, video capture device 112 may comprise a standalone video camera.

In some embodiments, the video capture device 112 may comprise a 2D camera configured to capture video. The video capture device 112 may be coupled to a shroud, such as a metal shroud, configured to fix or hold the video capture device 112 in a particular position such that it captures video from a fixed angle and/or orientation. The fixed angle and/or orientation may be selected to ensure that the video capture device 112 captures an appropriate or suitable view of the meat product. However, it will be appreciated that the use of a shroud is optional, and not necessary.

In some embodiments, the video capture device 112 may comprise a 3D camera configured to capture video data having depth information. In such embodiments, the 3D camera may comprise an Intel REALSENSE™ camera, or an equivalent camera.

Such a 3D camera may be configured to capture frames of the meat carcase from various differing angles and/or orientations. The use of a 3D camera can therefore remove any need for a shroud to maintain the fixed angle and/or orientation as mentioned above, and in some embodiments, for a static fixture 115. For example, the video capture device 112 may be deployed on a robotic arm (not shown), which may assist in the acquisition of frame over a range of angles and/or orientations. This can be particularly useful in meat processing conditions, which may include carcases moving on a chain, as the enablement of a greater set of angles and/or orientations may mean that suitable frame acquisition and/or accurate meat assessment can be maintained reliably as the carcases move along the chain.

Furthermore, use of a 3D camera for the video capture device 112 may not only allow for a range of frames from different angles and/or orientations to be captured, it may also allow for relatively high-resolution images to be captured, and accordingly, a greater amount of information that perhaps a 2D camera may allow for. This may improve the accuracy of downstream processes, such as selecting genetic stock, and determining suitable treatments for stock based on assessed traits.

In some embodiments, where at least one of the traits being assessed or measured is eye muscle area (EMA), marbling, ribeye area, rib fat thickness, meat colour, fat colour, marbling fineness and/or intramuscular fat proportion, use of a 3D camera may eliminate the need for a grid, such as a dot grid (e.g., a dot planimeter), to be placed or overlayed on a region of interest of the carcase such as the rib eye area to allow for determination of the area. Use of such a grid can be bothersome, and inefficient, requiring operator skill and time to be taken in placement, and sanitary practices to be implemented to ensure the grid is kept clean and intervention in moving it from carcase to carcase. Use of the 3D camera may be particularly advantageous where EMA is only one of a number of different traits being assessed, in which case, at least two separate video stream may need to be captured; one with the dot grid in place on the region of interest of the carcase, and another without the dot grid (which is not needed or unsuitable for assessing other traits).

In some embodiments, the video capture device 112 captures a video stream to better enable near real time processing and reduce memory storage impact.

As illustrated in FIG. 2, the analysis device 110 comprises a controller 202 configured to perform the analysis and output the results. To that end, the controller 202 may be in communication with the video capture device 112 and/or the user interface 120. The controller 202 comprises one or more processors 205 in communication with memory 210.

The processor(s) 205 may be arranged to retrieve data from memory 210 and execute program code stored within memory 210 to perform the described quality assessment functionality. The processor(s) 205 may include more than one electronic processing device and/or additional processing circuitry. For example, the processor(s) 205 may include multiple processing chips, a digital signal processor (DSP), analog-to digital or digital-to analog conversion circuitry, and/or other circuitry or processing chips that have processing capability to perform the functions described herein. The processor(s) 205 may execute all processing functions described herein locally on the analysis device 110.

Memory 210 may comprise a UI module 212, which, when executed by the processor 205, sends and receives instructions from the user interface 120, and allows for the output, such as the visual display and/or audio output, of information stored in memory 210 on the user interface 120.

Memory 210 comprises a meat quality assessment module 214, which, when executed by the processor(s) 205, determines meat quality assessment measure(s) for respective trait(s) of a meat product. In some embodiments, the meat quality assessment module 214 receives video data comprising a sequence of frames 104 as input. For example, at least some of the frames 104 may depict a part of a carcase to be quality assessed. The quality assessment module 214 may output a determined respective quality assessment measure for one or more traits relating to meat quality.

As mentioned above, the video capture device 112 may comprise a 2D camera or a 3D camera. In embodiments where the video capture device 112 comprises a 3D camera, the video capture device 112 may be external to or distinct from the analysis device 110. Memory 210 may comprise a 3D camera interface module 213. The 3D camera interface module 213 may comprise code libraries, which, when executed by the processor(s) 205, cause the 3D camera interface module 213 to send instructions to and receive instructions from the 3D camera. In embodiments utilising the depth functions of a 3D camera, such as when assessing traits such as EMA, marbling, ribeye area, rib fat thickness, meat colour, fat colour, marbling fineness and/or intramuscular fat proportion, processing depth data from the 3D camera may be performed by the camera interface module 213 and/or the meat quality assessment module 214. In some embodiments, the 2D camera may be used to assess traits including EMA, marbling, ribeye area, rib fat thickness, meat colour, fat colour, marbling fineness and/or intramuscular fat proportion. In some embodiments, video data obtained by the 2D camera may be assessed using non-depth modes of analysis. In some embodiments, the video data obtained by the 2D camera may be assessed using monocular depth estimation, stereo depth estimation from two different lenses, or similar techniques to perform a depth-based analysis using the 2D image(s) as a data source.

The meat quality assessment module 214 comprises an object determination module 216 and object quality determination module 218.

The object determination module 216 may comprise program code which, when executed by the processor 205, determines an area or region of interest 132 in a frame of the video data. Multiple areas or regions of interest 132 may be determined by the object determination module 216 for any given frame 104. For example, a different or distinct area or region of interest 132 may be determined for the assessment of each trait.

In some embodiments, the object determination module 216 may comprise one or more segmentation models 220. Each segmentation model 220 may be configured to determine an area or region of interest 132 within a frame for analysis. In some embodiments, a determined area or region of interest may be suitable for performing a meat quality assessment measure for more than one, or even all traits of interest. The segmentation model(s) 220 may be a machine learning model, trained on a data set of images of processed carcases and/or meat products, and configured to determine the presence, boundaries, and/or segmentation of meat product in a frame 104.

The segmentation model 220 may comprise a convolutional neural network. The segmentation model 220 may be configured to identify the region of interest 132 in a frame 104. After the region of interest 132 is identified, the segmentation model 220 may crop the frame 104 to focus on the proportions of the area of interest 132. In some cases, other parts of the image are masked out, and the cropped (and/or masked) frame 104 may be scaled. The cropped and/or masked and/or scaled frame 104 may be fed into a further convolutional network within segmentation model 220 to produce a scalar trait prediction.

When the trait being assessed is EMA, the segmentation model 220 may be configured to process frames featuring a planimeter positioned or placed on the area of interest of the carcase, such as the rib-eye area. In some embodiments, the segmentation model 220 may be configured to operate according to a first state or mode of operation for assessing traits other than EMA, and which do not require placement of a dot grid on the area of interest of the carcase when acquiring the video frames, such as marbling, ribeye area, rib fat thickness, meat colour, fat colour, marbling fineness or intramuscular fat proportion. The segmentation model 220 may be configured to operate according to a second state or mode of operation for assessing traits such as EMA, marbling, ribeye area, rib fat thickness, meat colour, fat colour, marbling fineness or intramuscular fat proportion, and which do require placement of a dot grid on the area of interest of the carcase when acquiring the video frames, such as for example, where a 2D camera is being used. The user interface 120 may be configured to allow the user to elect a desired mode of operations for segmentation model 220. For example, the user interface 120 may be configured to present or display a user interface element to allow a user to select a desired mode of operation for the segmentation model 220.

For the second mode of operation, the segmentation model 220 may be trained on meat product images with grids to identify the eye muscle and recognise the presence of planimeter dots within the frame 104.

In some embodiments, the object determination module 216 may comprise a MobileNetV3 encoder with Lite Reduced Atrous Spatial Pyramid Pooling segmentation decoder. In other embodiments, the object determination module 216 may comprise other machine learning model(s) such as DeepLabv3, Mask R-CNN, EAR-Net, etc. In some embodiments, a binary cross-entropy loss function is used. However, it will be appreciated that other loss functions such as Dice loss, or sparse categorical cross entropy could be used. In some embodiments, training data augmentation techniques may be used to augment or adjust the image, such as: rotation and/or flipping and/or contrast adjustment and/or brightness adjustment and/or cropping and/or resizing of the image.

In some embodiments, the segmentation model 220, when undertaking an EMA segmentation process, may comprise YOLOv4-tiny. The loss function used may be a generalized IoU loss model.

The object quality determination module 218 may comprise program code defining one or more trait prediction model(s), which, when executed by the processor 205, determine a prediction score for each respective trait based on the area or region of interest, as determined by the object determination module 216. Object quality determination module 216 may comprise a machine learning model, trained on a data set of images of processed carcases and/or meat products, and configured to determine traits such as marbling, marbling fineness, ribeye area, rib fat thickness, intramuscular fat, fat colour, and meat colour within images depicting meat products.

In some embodiments, the object quality determination module 216 may comprise a MobileNetV3 encoder with custom pooling. The encoder may utilise fully connected layers. In other embodiments, the object quality determination module 216 may comprise an EfficientNetv2 model. In other embodiments, the object quality determination module 216 may comprise an FBNet model. In still other embodiments, the object quality determination module 216 may comprise a ShuffleNetv2 model. The loss function used in the object quality determination module 216 may comprise a mean absolute error function. In other embodiments, the loss function may be a root-mean-square error function.

In some embodiments, the training data of the machine learning models used in object quality determination module 216 may be augmented by the rotation of input data. In some embodiments, the training data of the object quality determination module 216 may be augmented by flipping of input data. In some embodiments, the training data of the object quality determination module 216 may be augmented by changing the contrast of input data. In some embodiments, the training data of the object quality determination module 216 may be augmented by changing the brightness of input data. In some embodiments, the training data of the object quality determination module 216 may be augmented by changing the brightness of input data. In some embodiments, the training data of the object quality determination module 216 may be augmented by cropping of input data. In some embodiments, the training data of the object quality determination module 216 may be augmented by resizing input data. The augmentation of data in this way may create a more resilient and accurate data set.

The meat quality assessment module 214 comprises a frame suitability determination module 224. The frame suitability determination module 224 may be configured to determine whether an acquired frame of the video data is suitable or sufficient for assessing a particular trait of the meat product 140. In some embodiments, the frame suitability determination module 224 is configured to determine a frame suitability score for predicting each respective trait. Some frames may be suitable or sufficient for assessing a first trait in a meat product 140, but may be insufficient or unsuitable for assessing a second trait in the meat product 140. Accordingly, the frame suitability determination module 224 may apply different criteria and/or thresholds in determining frame suitability scores for the assessment of different traits. Similarly, the frame suitability determination module 224 may apply the same criteria and/or thresholds in determining frame suitability scores for the assessment of different traits. In some embodiments, the frame suitability determination module 224 may compare the frame suitability score for a trait to a threshold frame suitability score for the trait, and response to the frame suitability score being greater than the threshold frame suitability score for the trait, the frame suitability determination module 224 may determine the frame as a suitable frame for assessing or predicting the trait. Responsive to the frame suitability score being less than the threshold frame suitability score for the trait, the frame suitability determination module 224 may determine the frame as an unsuitable frame for predicting the trait. In some embodiments, the frame suitability determination module 224 may discard or discount the unsuitable frame for further assessment. However, it should be appreciated that a frame deemed unacceptable or unsuitable for assessing a first trait may nonetheless be suitable for assessing a second trait. In some embodiments, a frame deemed unacceptable for a first trait but suitable for a second trait may be discarded on the basis that it is unacceptable for the first trait. The threshold suitability scores may be configurable. The threshold suitability scores may be configured differently for each trait. For example, each trait may have trait specific threshold values; a frame that may be suitable for assessing a first trait may be unsuitable for assessing a trait.

The meat quality assessment module 214 may be configured to determine an assessment confidence rating for each trait. The assessment confidence rating may be based on the suitability and/or number of suitable frames acquired, and/or on the predictions scores determined for the suitable frames. In some embodiments, the meat quality assessment module 214 determines the assessment confidence rating for a trait by determining one or more of: (i) a number of determined suitable frames for the trait; and (ii) a function of the frame suitability scores for the trait of the determined suitable frames. In some embodiments, the meat quality assessment module 214 determines the assessment confidence rating for a trait by determining a function of the prediction scores for the first trait of the determined suitable frames.

The meat quality assessment module 214 may determine whether to determine or generate a quality assessment measure for the trait(s) based on the respective assessment confidence rating(s). For example, the meat quality assessment module 214 may compare the assessment confidence rating to a threshold value for the respective trait. If the assessment confidence rating exceeds the relevant threshold, the meat quality assessment module 214 may determine or generate the quality assessment measure for the trait. The quality assessment measure for the trait may be based on the output from the object quality determination module 218, such as the prediction score(s) for the trait(s) of the determined suitable frames. However, if the assessment confidence rating does not exceed the relevant threshold, the meat quality assessment module 214 may acquire and assess further frames of the video data for analysis.

The meat quality assessment module 214 may be configured to determine a carcase identifier. For example, the carcase identifier may be provided within the image frames 104. The carcase identifier may be on meat product 140. The carcase identifier may be in a nearby area to the meat product 140. The carcase identifier may be one or more of: a one-dimensional barcode; a two-dimensional barcode (such as a QR code, or DotMatrix system for example); an alpha numerical code; a near-field communication (NFC) tag; an RFID tag. In some embodiments, the carcase identifier may be entered via the user interface 120. In such embodiments, the user interface receives the carcase identifier and transmits it to the memory 210 via the processor 205. In some embodiments, the meat quality assessment module 214 sends a request to the processor 205 to retrieve a carcase identifier from an external application, using an application programming interface (API). The carcase identifier may be stored within memory 210. The carcase identifier may be stored within the meat quality assessment module 214. The meat quality assessment module 214 may associate the carcase identifier with an image frame 104. The meat quality assessment module 214 may associate the carcase identifier with a meat product 140. The use of a carcase identifier may allow for accurate retrieval of meat quality assessments, which correspond to specific carcases. The processor 205 may determine the carcase identifier through an application programming interface (API). The API may allow communication with a processing facility network where a carcase identifier is stored. The user interface 120 may comprise a display 130, configured to display the determined meat quality assessment measure(s). The user interface 120 may comprise a keyboard, touch screen, and/or button based interface. The display 130 may comprise an LED or LCD display, such as a smartphone touch screen display.

In some embodiments, the analysis device 110 may be mounted on a robotic arm (not shown). In such embodiments, the robotic arm (not shown) may be controlled by controller 202 to orient the video capture device 112 of the analysis device 110 to match, align or accommodate predetermined viewing angles of moving carcases in a meat processing environment, for example, to allow for consistent and/or relatively fast capture of video images by the analysis device 110. The robotic arm (not shown) may improve the accuracy and/or efficiency of the analysis process, as a robotic arm mount may be able to better match speed and orientation of carcases as they move during processing, and/or maintain a consistent perspective. Furthermore, such embodiments may allow for a reduction in labour cost for plants.

In some embodiments, the analysis device 110 may be installed as part of a meat slicing assembly (not shown). In such embodiments, the video capture device 112 of analysis device 110 may be oriented to capture video images of a facing of a meat primal. The output of the analysis device 110 can be used to determine the quality of facing of the meat primal. After a determination of the quality of the end of the meat primal is made, it can then be sliced by the slicing assembly (not shown) and directed along one or a plurality of different conveyors. For example, each conveyor may lead to a separate or different processing area, where the slices are processed, labelled and/or packaged according to their assessed meat quality. Accordingly, such embodiments allow for relatively rapid processing of meat products, and/or a relatively greater specificity of meat quality throughout an entire meat primal. This is an improvement over existing solutions of high volume meat processing, which typically assign a single meat quality grade for processed meat primals—which may then fail to capture higher quality portions which exist throughout the primal.

Communications interface 226 is accessible by the processor 205 and configured to allow exchange of information with devices external of the analysis device 110. The communications interface 226 may comprise components to receive a SIM card to facilitate communication through 2G, GSM, EDGE, CDMA, EVDO, 3G, GPRS, 4G, 5G or other suitable telecommunication networks. Communications interface 226 may comprise an Ethernet port to enable wired communication. Communications interface 226 may comprise a wireless internet interface. Communications interface 226 may comprise a Bluetooth interface. Communications interface 226 may comprise one or more of any of the described embodiments above. Communications interface 226 may enable communication through an API. Communications interface 226 may enable communication through hypertext transfer protocol (HTTP, HTTPS).

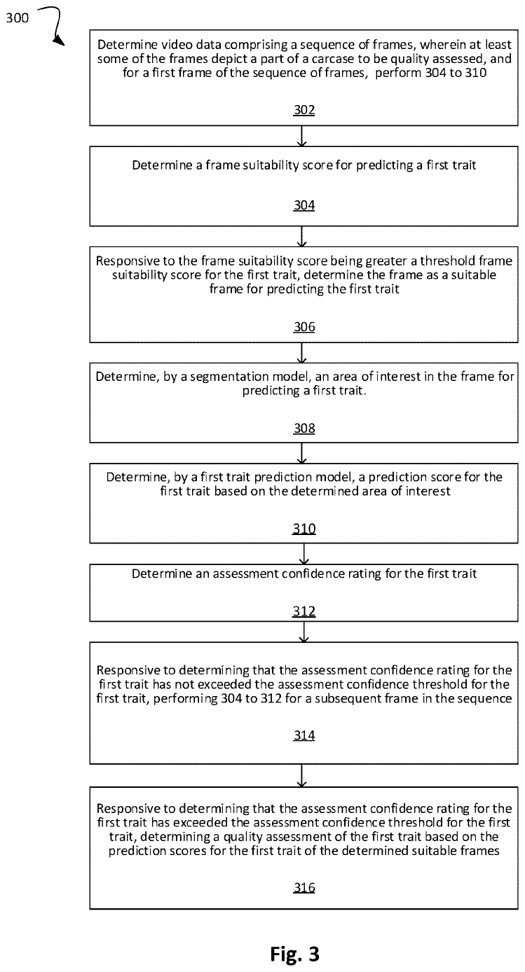

FIG. 3 is a flowchart of a method 300 of assessing meat quality according to some embodiments. The method 300 may be performed by the analysis device 110 executing program code in memory 210, such as the meat quality assessment module 214.

At 302, the analysis device 110 determines video data. The video data comprises a sequence of frames 104, or a series of images. At least some of the frames depict a part of a carcase or meat product 140 to be quality assessed. In some embodiments, the video data is captured by the video capture device 112 and provided to the meat quality assessment module 214. The sequence of frames 104 may be stored within memory 210 for assessment by the meat quality assessment module 214. The sequence of frames 104 may be stored temporarily and in near-real time during the method 300, to avoid the retention of large image files which may adversely impact the storage on memory 210. Accordingly, in some embodiments, each frame 104 of the video data may be analysed as it is received, and while the video data is still being captured by video capture device 112.

In some embodiments, the trait being assessed is EMA. However, it will be appreciated that the trait being assessed may include one or more of EMA, marbling, ribeye area, rib fat thickness, meat colour, fat colour, marbling fineness or intramuscular fat proportion. As mentioned above, the analysis device 110 may be configured to operate in a second operation mode, for example, as selected by a user providing an input to the user interface, or automatically on detection of the placement of a grid on the region of the carcase being captured. The second operation mode may be configured specifically for assessing EMA of a meat product 140 having a grid placed thereon such that the frames of the meat product 140 captured by the analysis device 110 also depict the grid. The grid may be a planimeter, such as a translucent or substantially translucent dot planimeter, for example. The grid allows the analysis device 110 to determine an area of the region of interest of the carcase.

At 304 to 312, the meat quality assessment module 214 undertakes a series of actions for a first image frame 104 for the sequence of frames.

At 304, the meat quality assessment module 214 determines a frame suitability score for assessing or predicting a first trait. The frame suitability score may relate to the suitability or appropriateness or sufficiency of a particular or candidate frame for assessing a particular trait. For example, higher-valued scores may indicate a greater degree of suitability for accurate prediction of that trait. The frame suitability score may be based on one or more frame suitability ratings.

In some embodiments, the frame suitability score may depend on a frame suitability-rating indicative of a lack of glare. For example, the meat quality assessment module 214 may be configured to convert the frame to greyscale. After the frame is converted to greyscale, the meat quality assessment module 214 may determine or calculate a number of pixels which exceed a configurable brightness value, as a proportion of the total number of pixels in the image. If the number of greyscale pixels exceeds the configurable brightness value (frame suitability rating threshold for lack of glare), then the frame may be determined to have too much glare to be used in assessing meat quality. Such frames may be discarded, or discounted from further analysis.

In some embodiments, the frame suitability score may depend on a frame suitability rating indicative of image sharpness. For example, the meat quality assessment module 214 may be configured to perform a fast Fourier transform (FFT) based method configured to measure high-frequency components of the image. If the high frequency components of the image are determined to correspond to an image of sufficient sharpness (frame suitability rating threshold for sharpness), then the frame may be used in the assessment of meat quality. Frames that do not have sufficient sharpness may be deemed too blurry to be accurately assessed for meat quality, and may be discarded or discounted for further analysis.

In some embodiments, the frame suitability score may depend on a frame suitability rating indicative of segmentation size. This rating measures the size of the identified region of interest 132 relative to the size of the entire frame. The meat quality assessment module 214, by the segmentation model 220, may determine the size of the identified segment as a number of pixels as a segment pixel score. If the segment pixel score is lower than a threshold, the frame may be determined to lack sufficient segmentation size, and may be discarded or discounted for further analysis. The frame suitability rating threshold for segmentation size may be a minimum ratio of the determined segment pixel score compared to the overall number of pixels in the frame.

In some embodiments, the frame suitability score may depend on a frame suitability rating indicative of segmentation margin. The segmentation margin is a rating that relates to the shortest distance between a segmented region of interest 132 and any edge of the frame. Values that are too small may indicate the region of interest 132 being cut off by the frame edge. If a frame is determined by meat quality assessment module 214 to have a segmentation margin score below a threshold (frame suitability rating threshold for segmentation margin), then the frame may be discarded or discounted for further analysis.

In some embodiments, the frame suitability score may depend on a frame suitability rating indicative of segmentation roundness. The segmentation margin is a rating that measures roundness of the region of interest 132. This may be performed by the meat quality assessment module 214 by fitting an ellipse to the area and taking its aspect ratio. Relatively highly oblique angles to the animal cut surface may be determined to be undesirable for prediction of some traits, and this value may be used as a proxy for non-obliqueness of the camera angle.

In some embodiments, the frame suitability rating may include the application or output of a frame suitability model or a frame quality model. The frame suitability model may include a neural network configured to receive input of a frame image. The neural network may be a convolutional neural network. The frame image may be segmented or unsegmented. In some embodiments, the frame image may be rescaled or not rescaled. The frame suitability model is configured to assess the quality or suitability of the frame and output a representative indicator. The representative indicator may be an indicator representing overall quality or suitability, a rating of quality or suitability, or different aspects of quality or suitability. In some embodiments, the frame suitability model is configured to output a plurality of representative indicators. The representative indicator may be numerical or categorical. In some embodiments, the assessment module 214 may be configured to discard frames on the basis of the output from the frame suitability model.

In some embodiments, the frame suitability model may utilise other frame suitability ratings, including those discussed herein, as inputs to provide an assessment of quality or suitability, such as frame suitability ratings indicating segmentation roundness, segmentation margin, segmentation size, image sharpness, lack of glare, and the like. In some embodiments, the frame suitability model may be used in combination with other frame suitability measures to identify or determine high quality or suitable frames. For example, frame suitability measures may relate to quality or content of the frame image, depending upon the use of the frame suitability model. In some embodiments, the frame suitability model may be used in combination with or alongside the per-frame trait prediction results. For example, to choose a particular frame (or frames) of the plurality of frames from the video data for downstream processing or for visual display.

For each animal trait, the relevance of each of the ratings described above varies, with, for example, glare being extremely detrimental to some traits but less impactful on others. As such, for each trait, trait-specific thresholds can be configured for each of the different ratings, where frames are considered to be unsuitable for accurate prediction of that trait and excluded from further processing if any one of those thresholds is not met.

In some embodiments, one or more of the ratings may be used to calculate an overall score or the frame suitability score for each frame with respect to each trait.

In some embodiments, the frame suitability score Sₜ for a frame for a particular trait t may be calculated as:

$${S}_{t} = {{\sum }_{r}}_{} \qquad max ( {v}_{r} {a}_{r}^{t} + {b}_{{r}^{′}}^{t} , 0 )$$

where: vᵣ is a rating value of rating-type r,

$${a}_{r}^{t}$$

is a scaling coefficient of rating-type r for trait t, and

$${b}_{r}^{t}$$

is an offset coefficient of rating-type r for trait t (with both

$${a}_{r}^{t} and {b}_{r}^{t}$$

being constant between frames). For each trait/rating-type pair (t, r), the coefficients

$${a}_{r}^{t} and {b}_{r}^{t}$$

may be chosen such that higher values are indicative of greater suitability for accurate prediction, and Sₜ is always non-negative. The scaling and offset coefficients for each rating-type may be specifically selected or configured for each trait. This allows for greater weighting to be placed on one or more of the rating-types than other(s), thereby allowing for different criteria to be applied when considering the suitability of a frame for assessing two different types of traits.

In some embodiments, a multiplicative approach may be taken. For example, the frame suitability score Sₜ for a frame for a particular trait t may be calculated as:

$${S}_{t} = {\prod }_{r} \qquad max ( {v}_{r} {a}_{r}^{t} + {b}_{r}^{t} , 0 )$$

In some embodiments, the frame suitability score Sₜ for a frame for a particular trait t may be calculated as the geometric mean of the

$$max ( {v}_{r} {a}_{r}^{t} + {b}_{r}^{t} , 0 )$$

components.

In some embodiments, the frame suitability score Sₜ for a frame for a particular trait t is calculated as the minimum or the maximum of the individual

$$max ( {v}_{r} {a}_{r}^{t} + {b}_{r}^{t} , 0 )$$

components.

In some embodiments, the frame suitability score Sₜ for a frame for a particular trait t is calculated as the median of the

$$max ( {v}_{r} {a}_{r}^{t} + {b}_{r}^{t} , 0 )$$

components.

In some embodiments, the individual ratings

$${v}_{r} {a}_{r}^{t} + {b}_{r}^{t}$$

may be included in any of the above embodiments for calculating the frame suitability score Sₜ for a frame 104, for a particular trait t, without first determining the maximum of each rating with zero to produce a non-negative value.

At 306, responsive to the frame suitability score being greater than a threshold frame suitability score, the meat quality assessment module 214 determines that the frame is a suitable frame for assessing or predicting the first trait. The threshold may be configurable depending on the trait of interest, as different traits may have different requirements in order for the frame 104 to be determined as suitable for assessing or predicting the trait. When operating in the second operation mode for assessing EMA, the analysis device 110 may determine a frame 104 as being suitable for EMA assessment by determining whether a dot grid is depicted in the frame 104. In some embodiments, the analysis device 110 may determine a frame 104 as being suitable for EMA assessment if the meat quality assessment module 214 detects an eye muscle in the frame 104. In some embodiments, the analysis device 110 may determine a frame 104 as being suitable for EMA assessment by determining the presence of both a dot grid and an eye muscle within the frame 104.

At 308, the meat quality assessment module 214 (for example, the object determination module 216 or the segmentation model 220) determines an area or region of interest 132 in the frame 104, for assessing or predicting the first trait. For example, a frame of the video data may be provided to the object determination module 216 as an input, and the object determination module 216 may provide, as an output, a numerical value(s) indicative of the area of region of interest. In some embodiments, the object determination module 216 may generate an amended image or frame depicting the region or area of interest 132 on the frame, for example, as an outlined or bordered region. An example of the amended frame is illustrated in FIGS. 4a and 4b.

When operating in the second operation mode for assessing EMA, the analysis device 110 may determine the region of interest 132 within frame 104 by determining an eye muscle boundary within frame 104. In some embodiments, the determination of the region of interest 132 may further comprise a determination of a dot grid overlayed over an eye muscle boundary within frame 104. In some embodiments, the segmentation model 220 may mask the frame 104 to remove the parts of the image that are not within the region of interest 132. In such instances, the masking of the frame 104 may reduce the chance that dots from the dot grid that fall outside of the determined region of interest are erroneously counted.

At 310, the meat quality assessment module 214 (for example, the object quality determination module 218 or the trait prediction model 222) determines a prediction score for the first trait based on the determined region or area of interest 132. The prediction score for the first trait may comprise a scalar-value for the first trait corresponding to the first frame 104.

When operating in the second operation mode for assessing EMA, the meat quality assessment module 214 may determine the number of dots within the identified region of interest 132. As each of the dots corresponds to a square centimetre, this determination provides a measurement of the eye muscle area in cm². In some embodiments, a different grid size may be used, such as a smaller grid or a larger grid. In embodiments where a smaller grid is used, dots may be less than one squared centimetre apart. In embodiments where a larger grid is used, dots may be more than one squared centimetre apart. In such embodiments, a user may select grid size via the user interface 120. In other embodiments, a user may select an area measurement that each dot corresponds to.

At 312, the meat quality assessment module 214 determines an assessment confidence rating for the first trait. The assessment confidence rating is indicative of whether sufficient information has been determined from the video data to generate a quality assessment measure for the first trait.

In some embodiments, the meat quality assessment module 214 determines the assessment confidence rating for the first trait by determining whether a sufficient or threshold number of suitable frames have been determined. If the total of the determined suitable frames does not exceed the threshold (a minimum), the meat quality assessment module 214 determines that the assessment confidence rating is not sufficient.

In some embodiments, the meat quality assessment module 214 determines the assessment confidence rating for the first trait based on a function of the frame suitability scores for the first trait of the determined suitable frames. For example, the frame suitability scores for all non-excluded frames may be summed or added together and compared to a threshold value (a minimum). If the total of the frame suitability scores does not exceed the threshold, the meat quality assessment module 214 determines that the assessment confidence rating is not sufficient.

In some embodiments, the meat quality assessment module 214 determines the assessment confidence rating for the first trait based on a function of the prediction scores for the first trait of the determined suitable frames. For example, the function of the prediction scores may be a sample standard deviation of the prediction score(s) for the determined suitable frame(s) for the trait. The sample standard deviation value may be compared with a threshold value (a maximum). If sample standard deviation value exceeds the threshold, the meat quality assessment module 214 determines that the assessment confidence rating is not sufficient.

In some embodiments, the meat quality assessment module 214 is configured to determine whether or not the frame is a suitable frame (for example, 304 and 306) and determine a prediction score for the frame (for example, 308 and 310) substantially simultaneously or substantially in parallel.

In some embodiments, the meat quality assessment module 214 only determines a prediction score for frames that have been first determined as suitable frames. For example, responsive to determining the frame as a suitable frame for predicting or assessing the first trait (for example at 304 and 306), the meat quality assessment module 214 determines a prediction score for the frame (for example, at 308 and 310). Responsive to determining the frame as not being a suitable frame for predicting or assessing the first trait, the meat quality assessment module 214 may elect not to determines a prediction score for the frame (for example, not perform 308 and 310).

At step 314, responsive to determining that the assessment confidence rating for the first trait has not exceeded the assessment confidence threshold for the first trait, the meat quality assessment module 214 performs 304 to 310 for a subsequent frame in the sequence of frames, as for example, may be received from the video capture device 112.

At step 316, responsive to determining that the assessment confidence rating for the first trait has exceeded the assessment confidence threshold for the first trait, the meat quality assessment module 214 determines a quality assessment measure of the first trait based on the prediction scores for the first trait of the determined suitable frames. Furthermore, at step 316, after the quality assessment measure of the first trait is determined, the meat quality assessment module 214 may send instructions to the processor 205 to end the capture of video through the video capture device 112. In embodiments, for example, where more than one trait is being assessed, the meat quality assessment module 214 may send instructions to the processor 205 to end the capture of video through the video capture device 112 after each or all of the traits quality assessment is completed. This allows video capture to be stopped without requiring user input. The meat quality assessment may be stored within memory 210. The meat quality assessment module 214 may send instruction to the processor 205, to transmit the meat quality assessment through communications interface 226. In some embodiments, image frames 104 stored within memory 210 may be transmitted through communications interface 226. Communications interface 226 transmit data to an external source. In this way, once sufficient frames for accurate assessment of a trait have been determined, no further frames are analysed for that trait. This means that once a sufficient number of suitable frames have been acquired to allow the device to make a suitable assessment of the meat quality, no more frames are acquired and analysed. This contrasts to a technique where by a predetermined number of frames are acquired and assessed, in which case, it may be determined that the acquired number of frames is in fact insufficient to allow the assessment to be performed to a sufficient accuracy, or too many frames are acquired, where in fact a subset of the frames would have been sufficient to allow the assessment to be performed to a sufficient accuracy.

In some embodiments, the meat quality assessment module 214 determines a quality assessment measure of the first trait using an outlier-exclusion and averaging procedure as follows:

A second and first percentile values, P_{B} and P_{A}, respectively, of the prediction scores are calculated (where P_{B}≤P_{A}). For example, the first and second percentile values may be the 25^{th} and 75^{th} percentile (first and third quartile) respectively. In some embodiments, the second and first percentile values, P_{B} and P_{A}, may be set as 20 and 80, respectively, or 10 and 90, respectively, or 40 and 60, respectively. However, it will be appreciated that any suitable values may be used.

Then, the inter-quartile range IQR=P_{A}−P_{B} is calculated.

Then, a lower bound Bₘᵢₙ is calculated by multiplying the inter-quartile range by a tolerance parameter tol_{IQR} (configurable, with default value of 1.5) and subtracting this from the 25^{th} percentile value:

$${B}_{min} = {P}_{B} - ( {tol}_{IQR} \times IQR )$$

Similarly, an upper bound Bₘₐₓ is calculated as:

$${B}_{max} = {P}_{A} + ( {tol}_{IQR} \times IQR )$$

Finally, the arithmetic mean is taken over all individual-frame prediction values which fall in the range [Bₘᵢₙ, Bₘₐₓ] to produce the quality assessment measure of the first trait.

In some embodiments, in determining the quality assessment measure, the meat quality assessment module 214 may determine and ignore or disregard prediction scores derived from the ‘worst’ p_{drop} percent of frames based on the frame suitability ratings (for example, for each of glare, sharpness and segmentation size). In other words, in such embodiments, those prediction scores are not used to determine quality assessment measure for the trait.

In some embodiments, the quality assessment measure may be determined by an average of prediction scores taken without exclusion. In some embodiments, the quality assessment measure may be determined by taking the median or geometric mean of the prediction scores (as opposed to the arithmetic mean). In some embodiments, the quality assessment measure may be determined by taking a weighted average using the corresponding suitability scores; this may allow for frames 104 with higher suitability scores to have more influence over the resultant combination. In some embodiments, outliers may be excluded from the quality assessment measure by determining which multiple of the (sample) standard deviation each prediction is from the mean, and comparing those with a suitable threshold. In some embodiments, the quality assessment measure may be determined by clustering the predictions and selecting a determined cluster according to a criteria. The criteria may be the largest cluster, or a number of the largest clusters, such as the largest n clusters.

Once the meat quality assessment module 214 has determined the quality assessment measure, it may output the result to the user via the user interface 120. For example, the UI module 212 may cause presentation of the quality assessment measure for one or more traits on the display 130 and/or may store the quality assessment measure(s) in memory 210.

When all of the animal traits have reached this point, the video capture from the video capture device 112 may be ended by processor 205, and the user may be prompted to move to the next carcase for analysis by an alert at the user interface 120.

In some embodiments, an option to set a maximum number of frames NF for analysis may be included (which applies across all traits and includes all frames including ones which have been excluded from processing for one or more traits). If the total number of frames analysed exceeds this number, the first frame in the received sequence (the oldest frame) is dropped from future calculations and only the most recent NF frames are used for those traits for which a quality assessment measure has not already been produced.

In some embodiments, the meat quality assessment module 214 may assess the frames for a second trait and/or additional traits according to method 300. The second trait and/or additional traits are different from the first trait, and from each other. This assessment using the method 300 may be performed substantially simultaneously to the assessment of the first trait using method 300.

As mentioned above, in some embodiments, the video capture device 112 may comprise a 3D camera configured to determine depth information, which may be used in assessing the trait of EMA. However, it will be appreciated that the depth information may also be used in assessing the traits of marbling, ribeye area, rib fat thickness, meat colour, fat colour, marbling fineness or intramuscular fat proportion. The processor 205 may receive instructions from the user interface 120 to initiate the 3D camera to record 3D video data. The 3D video data may comprise video image frames 104 taken from more than one orientation. The 3D video image data comprising video image frames 104 taken from more than one orientation may be processed by 3D camera interface module 213. The 3D camera interface module 213 may determine a 3D point-cloud based on the captured 3D video data. The 3D camera interface module 213 may cross-reference the image frame 104 to produce a 3D model of an object in the camera's field of view. This may comprise a meat product 140. In some embodiments this may comprise a portion of a meat product 140. The 3D camera interface module 213 may communicate with the meat quality assessment module 214. The 3D camera interface module may send the 3D point cloud to the object determination module 216. The object determination module 216 may receive the point cloud from the 3D camera interface module 213 and isolate the area of the point cloud corresponding to the region of interest 132. In some embodiments, another target area of the meat product 140 may be identified in the point cloud. This may comprise the region of interest 132. The 3D camera interface module 213 may send the 3D point cloud to the meat quality assessment module 214. The meat quality assessment module 214 may determine a plane, fitted to the points of the 3D-point cloud. The meat quality assessment module 214 may project the points of the 3D-point cloud onto the plane. This may generate a plurality of projected planar points which may be further processed. The area of the plane may be determined by the meat quality assessment module 214 by the placement of the projected points on the plane. The determined area of the region of interest 132 may correspond to the eye muscle area.

Projected planar points may then be rasterized to a high-resolution image where each pixel cell of the image corresponds to a square region of the plane of known true size, that is a real-world area. For example, the real-world area may be determined by the meat quality assessment module 214 based on the video data. In some embodiments, the size of the pixel cell may be between 0.5 mm² and 0.01 mm². In some embodiments, the size of the pixel cell may be 0.25 mm². The square region of the plane of known true size may contain “holes”, that is, pixel cells within the region of interest which do not contain a corresponding point in the planar projection of the point cloud. The high-resolution image may then be converted to binary, in which every pixel has a value of either 1 or 0 depending on whether it contains a point in the planar projection. The resulting binary image may identify the area of the region of interest.

The meat quality assessment module 214 may then be configured to fill the holes in the image using a morphological closing operation. For example, a dilation operation followed by an erosion operation may be applied which uses the same structuring element for both operations. The morphological operation may use a kernel of appropriate size. The morphological closing operation may be effective for filling small holes in the image while preserving the shape and size of any large holes and objects in the image. The meat quality assessment module 214 may then use contour detection or contour recognition to obtain the border of the region of interest in the filled-in binary image. In some embodiments, the detection of a single contour is confirmed and/or verified. That is, where the filled-in region of interest is confirmed to be contiguous in the binary image, a number of possible failure modes are filtered out. Responsive to a single contour being identified, the known correspondence of the pixels to the real-world area is used to take the area inside the contour and convert it to a real-world region of interest area estimate.

Accordingly, the meat quality assessment module 214 may determine the size of the eye muscle area (EMA) based on the determined area of the region of interest 132. As the cut surface of a meat product 140 may be assumed to be approximately planar, errors due to noise or depth measurement may be corrected based on the approximately planar surface.

In some embodiments, the video capture device 112 may comprise a 3D camera configured to determine depth information, which may be used in assessing the trait of rib fat thickness. The meat quality assessment module 214 may be configured to measure the trait of rib fat thickness. Similar to the process for isolating the region of interest, the 3D camera interface module 213 may communicate with the meat quality assessment module 214, and may send a determined 3D point cloud to the object determination module 216. The object determination module 216 may receive the point cloud from the 3D camera interface module 213 and isolate both the areas of the point clouds corresponding to the eye muscle (EMA) and the area of the point cloud corresponding to the rib fat. In some embodiments, the EMA and rib fat points are isolated in the 3D point cloud by referencing a neural-network-derived segmentation of the colour image component of a depth map camera frame of the video data, where each pixel corresponds to a point in the 3D point cloud once cross-referenced with a depth map image. In some embodiments, isolating the EMA and the rib fat may be performed sequentially or in parallel.

The 3D camera interface module 213 may send the 3D point cloud to the meat quality assessment module 214. The meat quality assessment module 214 may determine a plane, fitted to the points of the 3D-point cloud. The meat quality assessment module 214 may project the points of the 3D-point cloud onto the plane. This may generate a plurality of projected planar points which may be further processed. The area of the plane may be determined by the meat quality assessment module 214 by the placement of the projected points on the plane. In this case, the determined area corresponds to the EMA and the rib fat. The plurality of projected planar points represent a 2D point cloud with both EMA and rib fat points. As these have been isolated separately from one another, the projected points for the EMA are distinct from the projected points of the rib fat.

The first principal component (PC1) of the EMA is identified and the 2D point cloud is aligned so that the PC1 is vertical. In some embodiments, it is assumed that the camera is held the right way up and that the filming occurs from the bottom 180 degrees of the EMA. This may be used to choose which of the two possible “ways up” it should be.

Projected planar points may then be rasterised into an image. In some embodiments, a coarser grid of pixel cells is used than that which is used in the process of calculating the are of the region of interest. For example, a 1 mm² pixel cell is used. The size of the pixel cells used may be chosen to minimise or reduce the number of holes. In some embodiments, the coarseness of the grid of pixel cells may be selected such that it is coarse enough that no holes are expected. That is, each pixel may contain or encompass at least one point from the planar projection. This provides a lower resolution 2D image (colour, not binary) with both the EMA and rib fat, where it is known which pixels correspond to the EMA and which pixels correspond to rib fat, and where the correspondence between pixels and real-world geometry is known.

In some embodiments, the method for measuring rib fat thickness may vary depending on the jurisdiction in which the method is being performed.

To measure the rib fat thickness, the y-axis row is identified at an appropriate percentage of the distance between the bottom and top of the EMA that meets the requirements of measuring the rib fat thickness. The top of the EMA may be aligned vertically upright along its first principal component, as described earlier. In some embodiments, the rib fat thickness may be measured approximately three quarters of the way up the EMA towards the top. In this case, the value of the y-axis row may be approximately 75%. In some embodiments, it may be within the range of 70 to 85%. In some cases, the value of the y-axis row may be 79%. However, it will be appreciated that this value may be empirically determined and may vary depending on the video data and the meat product.

The meat quality assessment module 214 may then be configured to measure the thickness of the rib fat along the y-axis row, and a predetermined number of surrounding rows around the y-axis row. For example, the rows between 74% and 83% of the way up the EMA. In some embodiments, the surrounding rows may be determined on the basis of a preselected threshold or buffer. The number of surrounding rows below the y-axis row value may be different to the number of surrounding rows above the y-axis row value. In some embodiments, the thickness may be measured in pixels. In some embodiments, the number of surrounding rows along which the thickness of the rib fat is measured may be determined empirically and may vary depending on the video data and the meat product.

The pixel thicknesses may then be converted back to real-world thickness estimates for the rib fat. The outlier-exclusion and averaging procedure described herein may then be used to aggregate the rows into an overall estimate for that frame. In some embodiments, the frames themselves are aggregated using a similar procedure. In some embodiments, the frames may be aggregated subject to suitability filtering, for example, where only frames determined to meet a particular suitability rating are aggregated. In some embodiments, where the difference between the largest and smallest per-row thickness estimates is too large within a given frame, the frame may be rejected for rib fat thickness measurement. In some embodiments, where too many of the rows within the percentage range being used do not contain any rib-fat points, the frames may be rejected for rib fat thickness measurement.

It will be appreciated that similar procedures to those described herein with reference to EMA and rib fat thickness may also be used to determine measurements relating to other traits such as marbling, ribeye area, meat colour, fat colour, marbling fineness or intramuscular fat proportion. In some embodiments, EMA marbling, ribeye area, rib fat thickness, meat colour, fat colour, marbling fineness or intramuscular fat proportion, may be assessed by analysing video data from the 2D camera or the 3D camera. In some embodiments, EMA, marbling, ribeye area, rib fat thickness, meat colour, fat colour, marbling fineness or intramuscular fat proportion may be assessed using depth and/or non-depth modes of analysis.

FIGS. 4a and 4b are example screenshots of displays of a user interface of the analysis device of FIG. 2, according to some embodiments. FIG. 4a depicts the display 130 having a frame of video data 402 depicting at a part of a carcase to be quality assessed. The video data 402 may be a live output from the video capture device 112. The video data 402 may be depicted with segments 410, 412 representing output of the segmentation model 220. Segments 410 may represent a determined segment corresponding to a fat portion of the carcase. Segments 412 may represent a determined meat product 140 on the carcase. In the embodiment of FIG. 4a and FIG. 4b, the determined meat product 140 is a rib eye cut. The segments to be determined may be shown adjacent the video data 402, as shown in 404 on the display 130. 406 may comprise at least one progress indicator of the confidence threshold for animal traits being assessed. 406 may be a progress bar 406 depicting a graphical representation of the progress of the number of determined suitable frames for each trait being assessed. In some embodiments 406 is a percentage indicator. In some embodiments 406 is numeral indicator. In some embodiments 406 is a word indicator.

At 408, the display 130 may depict operational icons that allow the display 130 to provide the functions of user interface 120, such as on-screen buttons to control the operation of the analysis device 110, or to provide the ability to control the operation of the meat quality assessment module 214. For example, in FIG. 4a, the analysis device 120 may be in the middle of performing method 300. Accordingly, the operational buttons at 408 allow for the display 130 to act as a user interface 120 to enable a user to stop the meat assessment operation. In the embodiment of FIG. 4b, which depicts the same carcase portion after the meat assessment is completed, the operational buttons at 408 allow for the re-initialisation of the method 300 on a new carcase, the flagging of a video for further assessment, or the changing of assessment modes (for example, between EMA and normal operation). Accordingly, as FIG. 4b depicts a completed meat quality assessment, 406 depicts the completed assessment for the respective animal traits. In the embodiment of FIG. 4b, these are marbling (both Australian and MSA grades), meat colour, fat colour, and intramuscular fat percentage. By presenting the part of the carcase being assessed 402, displaying the segmentation portions 412, 410, having context sensitive operational buttons at 408, and the progress/result of the meat quality assessment in 406, 418, a user can easily and effectively operation an analysis device 110 to perform the method 300. These operation and display of the user interface features depicted in FIGS. 4a and 4b are provided by executable code stored in the UI module 212.

In the embodiments of FIGS. 4a and 4b, the user interface is optimised to work with a touchscreen display, such as a smartphone display. However, in other embodiments, the display 130 may be a simple display screen without touch screen functionality. In such embodiments, the operational buttons at 408 and 416 may be physical buttons as part of analysis device 110. Alternatively, more than one display 130 may be provided, to display different information—such as the progress indicators of 406, or the result of the meat quality assessment in 418. In some embodiments, this information may be presented as part of the user interface 120 by lights, LCD screens, or other visual indicators to provide an operator with an indication of the outcome of the method 300 for given animal traits.

In some embodiments, the 3D point clouds generated by the 3D depth camera may be used to take a frame or a set of frames, captured from any angle to the cut surface of a meat product, to generate a high-resolution synthetic view of the cut surface. For example, as if it had been filmed from a top down, perpendicular or bird's eye view. In some embodiments, the angle to the cut surface may be within a reasonable range. In some embodiments, the 3D point clouds may also be used to generate high resolution synthetic views of the EMA, or EMA and rib fat thickness measurements, ribeye area, meat colour, fat colour, marbling fineness or intramuscular fat proportion. In some embodiments, video data captured by a 2D camera (or one or more frames from the video data) may be used to create a high-resolution synthetic image of a top down view. In some embodiments, a sequence of high-resolution synthetic images of a top down view of the cut surface of the meat product may be generated using the video data obtained by the 2D camera. In some embodiments, a neural network and/or machine learning approach may be used to generate the images, for example, by interpolating, extrapolating, or generating a synthetic image of the cut surface based on the obtained video data. In some embodiments, the high-resolution synthetic image (or a sequence of high-resolution synthetic images) may be created using a combination of video data obtained from both the 3D and 2D cameras. That is, the synthetic image of the cut surface may be created using both depth video data and normal video data.

The high resolution synthetic image may be generated directly on the analysis device 110 which includes the video capture device 112. In some embodiments, the synthetic image may be generated outside of the analysis device 110, for example, on a server, where the point cloud data is transmitted to an external server, and then configured to receive the generated synthetic image from the server. The synthetic image may be generated in real time during data capture, or after data capture has been performed.

The generation of the synthetic image from the point cloud data may include taking the low-resolution rasterised image as was output as a result of determining the rib fat thickness measurements, and applying upscaling techniques to take it from the low resolution imposed by the source pixel grid to a high resolution. In some embodiments, applying upscaling techniques may include using a generative ML model. Generating the synthetic image from the point cloud data may include taking the planar-projection 2D point cloud and applying one or more interpolation techniques before converting the point cloud to a high-resolution rasterised image using a finer pixel grid. In some embodiments, the interpolation techniques may be traditional or machine learning based techniques. In some embodiments, generating the synthetic image may include taking a higher-resolution rasterised image as was output as a result of determining the region of interest area as described herein, that was obtained using a finer-pixel grid, and “filling in” the holes with an inpainting technique. In some embodiments, the inpainting technique may use traditional or machine learning methods, for example a diffusion machine learning model.

In some embodiments, generating the synthetic image may include taking the 3D point cloud (or the isolated EMA or EMA and rib fat thickness points, or any other isolated part or trait points within the point cloud) and using a direct means to obtain a rasterised image at a high resolution. For example, a neural network may be used to obtain a rasterised image of the 3D point cloud at a high resolution. In some embodiments, generating a synthetic image may include taking the 2D planar projection point cloud (or the isolated EMA or EMA and rib fat thickness points, or any other isolated part or trait points within the point cloud) and using a direct means to obtain a rasterised image of the 2D point cloud at a high resolution. For example, a neural network may be used to obtain a rasterised image at a high resolution. In some embodiments, generating the synthetic image may include using any of the techniques described herein, whilst taking into account more than one frame at a time to produce a single resulting high-resolution synthetic image.

In some embodiments, all frames in the video data may be used to produce the single resulting high-resolution image. Using a plurality of frames may take advantage of information about the temporal sequence of frames used in the process. In some embodiments, a plurality of frames may be used without using the ordering of the frames or temporal information. The generated high-resolution synthetic image may be in the form of a sequence of frames and/or a video of high-resolution images each derived from one frame. In some embodiments, generating the synthetic image may include using a combination of two or more techniques described herein.

The generated high-resolution synthetic image may be used for further downstream processing. For example, for additional trait assessment/analysis. This further processing may be performed on the analysis device 110 or external to the analysis device 110. The generated high resolution synthetic image may be used for visual display. For example, either on the analysis device 110 or external to the analysis device 110, such as through some hardware accessory equipped with a display, or through an application such as an online web portal. In some embodiments, the synthetic image may be displayed in real-time during data capture, or subsequent to data capture.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A method comprising:
determining video data comprising a sequence of frames, wherein at least some of the frames depict a part of a carcase to be quality assessed;
for a first frame of the sequence of frames:
a) determining a frame suitability score for assessing a first trait;
b) responsive to the frame suitability score being greater than a threshold frame suitability score for the first trait, determining the frame as a suitable frame for assessing the first trait;
c) determining, by a segmentation model, a region of interest in the frame for assessing the first trait;
d) determining, by a first trait prediction model, a prediction score for the first trait based on the determined region of interest; and
e) determining an assessment confidence rating for the first trait;
responsive to determining that the assessment confidence rating for the first trait has not exceeded the assessment confidence threshold for the first trait, performing steps a) to e) for a subsequent frame in the sequence; and
responsive to determining that the assessment confidence rating for the first trait has exceeded the assessment confidence threshold for the first trait, determining a quality assessment measure of the first trait based on the prediction score for the first trait of the determined suitable frames.

2. The method of claim 1, wherein determining the assessment confidence rating for the first trait comprises:
determining one or more of: (i) a number of determined suitable frames for the first trait; and (ii) a function of the frame suitability scores for the first trait of the determined suitable frames.

3. The method of claim 1, wherein determining the assessment confidence rating for the first trait comprises:
determining a function of the prediction scores for the first trait of the determined suitable frames.

4. The method of claim 3, wherein determining a function of the prediction scores for the first trait of the determined suitable frames comprises determining an arithmetic mean taken over an interval:
$$[ {B}_{min} , {B}_{max} ]$$
where Bₘᵢₙ is defined by the function:
$${B}_{min} = {P}_{B} - ( {tol}_{IQR} \times IQR )$$
and where Bₘₐₓ is defined by the function:
$${B}_{max} = {P}_{A} + ( {tol}_{IQR} \times IQR )$$
where P_{A} is a prediction value of a first percentile of prediction scores of the first trait;
where P_{B} is a prediction value of a second percentile of prediction scores of the first trait;
where tol_{IQR} is a tolerance parameter; and
where IQR is an inter quartile range defined by
$$IQR = {P}_{A} - {P}_{B} .$$

5. The method of claim 4, further comprising:
excluding the lowest p_{drop} percentage of prediction scores for one or more of: (i) a measure of lack of glare; (ii) a measure of sharpness; and (iii) a measure of segmentation size;
where p_{drop} is a configurable parameter.

6. The method of claim 4 further comprising:
determining a frame number threshold for the sequence of frames;
responsive to determining the number of frames exceeds the frame number threshold, discarding the frame suitability score, prediction score, and confidence rating for the earliest determined frame.

7. The method according to claim 3, wherein the first percentile is a smaller percentile than the second percentile.

8. The method of claim 1, further comprising:
responsive to determining the frame as a suitable frame for assessing the first trait, performing steps c) and d); and
responsive to determining the frame as not being a suitable frame for assessing the first trait, omitting steps c) and d).

9. The method of claim 1, wherein determining video data comprising receiving a video stream.

10. The method of claim 1, wherein the frame suitability score is based on one or more of: (i) a measure of lack of glare, (ii) a measure of sharpness, (iii) a measure of segmentation size; (iv) a measure of segmentation margin of the region of interest; (v) a measure of segmentation roundness of the region of interest.

11. The method of claim 1, wherein determining the frame suitability score comprises determining the frame suitability score according following equation:
$${S}_{t} = {\sum }_{r} \qquad max ( {v}_{r} {a}_{r}^{t} + {b}_{r}^{t} , 0 ) ,$$
where Sₜ is the suitability score;
vᵣ is a value of rating-type r for the first frame;
$${a}_{r}^{t}$$
is a scaling coefficient of the rating type r for trait t; and
$${b}_{r}^{t}$$
is an offset coefficient of rating r for trait t.

12. The method of claim 1, further comprising:
outputting the quality assessment measure of the first trait to a user interface.

13. The method of claim 1, wherein the first trait comprises any one of: eye muscle area, marbling, marbling fineness, ribeye area, rib fat thickness, intramuscular fat, fat colour, and meat colour.

14. The method of claim 1, further comprising:
for the first frame of the sequence of frames:
f) determining a frame suitability score for assessing a second trait, wherein the second trait is different from the first trait;
g) responsive to the frame suitability score being greater a threshold frame suitability score for the second trait, determining the frame as a suitable frame for assessing the second trait;
h) determining, by a second segmentation model, area region of interest in the frame for assessing a second trait;
i) determining, by a second trait prediction model, a prediction score for the second trait based on the determined region of interest; and
j) determining an assessment confidence rating for the second trait;
responsive to determining that the assessment confidence rating for the second trait has not exceeded the assessment confidence threshold for the second trait, performing steps a) to e) for a subsequent frame in the sequence; and
responsive to determining that the assessment confidence rating for the second trait has exceeded the assessment confidence threshold for the second trait, determining a quality assessment measure of the second trait based on the prediction scores for the second trait of the determined suitable frames.

15. The method of claim 14, wherein the second trait is different from the first trait and comprises any one of: eye muscle area, marbling, marbling fineness, ribeye area, rib fat thickness, intramuscular fat, fat colour, and meat colour.

16. The method of claim 1, wherein the frame suitability score is based on one or more frame suitability ratings, each frame suitability rating being indicative of the suitability of a frame with respect to a specific measure, the method further comprising, for each of the one or more frame suitability ratings:
comparing the frame suitability rating with a respective measure specific rating threshold; and
responsive to determining that the frame suitability rating does not meet the threshold, determining the frame as unsuitable and excluding the frame from further processing.

17. A meat assessment system, comprising:
at least one processor;
memory accessible to the at least one processor and comprising computer executable instructions, which when executed by the at least one processor, causes the system to perform operations including:
determining video data comprising a sequence of frames, wherein at least some of the frames depict a part of a carcase to be quality assessed;
for a first frame of the sequence of frames:
a) determining a frame suitability score for assessing a first trait;
b) responsive to the frame suitability score being greater than a threshold frame suitability score for the first trait, determining the frame as a suitable frame for assessing the first trait;
c) determining, by a segmentation model, a region of interest in the frame for assessing the first trait;
d) determining, by a first trait prediction model, a prediction score for the first trait based on the determined region of interest; and
e) determining an assessment confidence rating for the first trait;
responsive to determining that the assessment confidence rating for the first trait has not exceeded the assessment confidence threshold for the first trait, performing steps a) to e) for a subsequent frame in the sequence; and
responsive to determining that the assessment confidence rating for the first trait has exceeded the assessment confidence threshold for the first trait, determining a quality assessment measure of the first trait based on the prediction score for the first trait of the determined suitable frames.

18. The system of claim 17, further comprising:
a user interface configured to display the determined quality assessment measure for the first trait.

19. The system of claim 17, further comprising a video capture device to capture video data, controlled by the at least one processor.

20. A non-transitory machine-readable medium storing instructions which, when executed by one or more processors, cause an electronic apparatus to perform operations including:
determining video data comprising a sequence of frames, wherein at least some of the frames depict a part of a carcase to be quality assessed;
for a first frame of the sequence of frames:
a) determining a frame suitability score for assessing a first trait;
b) responsive to the frame suitability score being greater than a threshold frame suitability score for the first trait, determining the frame as a suitable frame for assessing the first trait;
c) determining, by a segmentation model, a region of interest in the frame for assessing the first trait;
d) determining, by a first trait prediction model, a prediction score for the first trait based on the determined region of interest; and
e) determining an assessment confidence rating for the first trait;
responsive to determining that the assessment confidence rating for the first trait has not exceeded the assessment confidence threshold for the first trait, performing steps a) to e) for a subsequent frame in the sequence; and
responsive to determining that the assessment confidence rating for the first trait has exceeded the assessment confidence threshold for the first trait, determining a quality assessment measure of the first trait based on the prediction score for the first trait of the determined suitable frames.
